(19) United States
(12) Patent Application Publication
(10) Pub. No.: US 2026/0100243 A1
(43) Pub. Date: Apr. 9, 2026

(54) SYSTEM AND METHOD FOR GENERATING SEQUENCES FOR THERAPEUTIC PROTEINS

(71) Applicant: LG MANAGEMENT DEVELOPMENT INSTITUTE CO., LTD., Seoul (KR)

(72) Inventors: Kiyoung KIM, Seoul (KR); Soorin YIM, Seoul (KR); Doyeong HWANG, Seoul (KR)

(21) Appl. No.: 19/417,417

(22) Filed: Dec. 12, 2025

Related U.S. Application Data
(63) Continuation of application No. PCT/KR2025/095417, filed on Jun. 16, 2025.

(30) Foreign Application Priority Data
Sep. 5, 2024 (KR) 10-2024-0120899

(51) Int. Cl. G16B 15/00; G16B 40/00

(52) U.S. Cl. CPC G16B 15/00; G16B 40/00

(57) ABSTRACT

A system and method for generating protein amino acid sequences having a user-desired property are provided. Using a noise-based diffusion model, the system and method can generate amino acid sequences of proteins that have excellent disease treatment effects and are safe for use as therapeutic agents in a human body. The system can function by obtaining reference protein sequence information, generating noise-added protein sequence information, iteratively generating noise-removed protein sequence information and partially noise-added protein sequence information, and generating noise-removed output protein sequence information. Noise may be added to protein sequence information using a Gaussian or other known noise model. Noise may be removed from protein sequence information using an artificial neural network model trained by a method of minimizing a loss function. By incorporating sequence guidance and structure guidance derived from known proteins, users can generate improved candidate protein drugs for testing.

### CROSS REFERENCE TO RELATED APPLICATIONS

This application is a Bypass Continuation of International Patent Application No. PCT/KR2025/095417, filed on June 16, 2025, which claims priority from and the benefit of Korean Patent Application No. 10-2024-0120899, filed on September 5, 2024, which is hereby incorporated by reference for all purposes as if fully set forth herein.

### BACKGROUND

### FIELD

Embodiments of the invention relate generally to a system and a method for generating protein amino acid sequences having a user-desired property. More particularly, the present disclosure relates to a system and a method for generating amino acid sequences of proteins that have excellent disease treatment effects and are safe in a human body to be used as therapeutic agents.

### DISCUSSION OF THE BACKGROUND

Recently, as artificial intelligence technology has been developed, efforts to shorten research and development periods and to increase efficiency by utilizing artificial intelligence technology have been continuously made in the field of new protein therapeutic agent development. Attempts have been continuously made to generate protein amino acid sequences that are predicted to have a property of binding to a target protein as requested by a user by training an artificial intelligence model with accumulated data on amino acid sequences of proteins and structures, physical properties, functions of the proteins, and the like. However, in order to develop new protein therapeutic agents, in addition to the property of binding to the target, human safety must also be sufficiently considered, but research on providing an artificial intelligence model for generating therapeutic proteins with sufficient consideration of human safety has been very insufficient so far.

Since therapeutic protein drugs have large sizes compared to small molecule drugs including traditional substances chemically synthesized as active ingredients, the therapeutic protein drugs may cause unexpected side effects upon human immune cells. Such unexpected side effects may be fatal, and, therefore, pharmaceutical companies developing therapeutic protein drugs must predict side effects before therapeutic protein drugs are administered to humans and design therapeutic protein drugs having excellent human safety. Accurate prediction of such side effects before actually administering the therapeutic protein drugs to humans remains a difficult task, and, thus, considerable cost and time are required to evaluate human safety.

Accordingly, there is a need in the art for an artificial intelligence system and method capable of generating therapeutic proteins having excellent human safety as well as excellent therapeutic effects by sufficiently considering side effects that may occur when they are administered to a human body.

The above information disclosed in this Background section is only for understanding of the background of the inventive concepts, and, therefore, it may contain information that does not constitute prior art.

### SUMMARY

The present disclosure is directed to providing a system and method for generating protein sequence informationdefining new proteins that show improvement in properties specified by a user.

The present disclosure is directed to providing a system and method for predicting physical properties, structures, binding affinities, and interaction states of proteins from protein data or for designing amino acid sequences of proteins having desired properties. The present disclosure is further directed to providing a system and method having higher protein property prediction reliability in comparison with conventional systems.

Additional features of the inventive concepts will be set forth in the description which follows, and in part will be apparent from the description, or may be learned by practice of the inventive concepts.

One embodiment of the present disclosure may provide a protein sequence generation system for new therapeutic protein drug development.

The present disclosure may provide a system including equipment for synthesizing a protein, a memory configured to store one or more instructions, and at least one processor configured to execute the one or more instructions stored in the memory.

Operations performed by the one or more instructions may include a step of obtaining reference protein sequence information, a step of generating noise-added protein sequence information by repeatedly adding noise to the reference protein sequence information, and a step of generating noise-removed output protein sequence information from the noise-added protein sequence information, wherein the steps of the generating may include a step of generating the noise-removed protein sequence information removed by removing noise from input protein sequence information according to one or more protein structure guidances specified by a user, and a step of repeatedly performing all or part of a step of generating the noise-added protein sequence information by adding noise to the noise-removed protein sequence information according to one or more protein sequence guidances specified by the user,

wherein all or part of the steps of the generating are repeatedly performed,

wherein the noise-removed output protein sequence information resulting from a final iteration of the generating steps is used with the equipment to synthesize a candidate protein, and

wherein, in relation to the reference protein, the candidate protein exhibits improved properties corresponding to the protein structure guidances.

The system may generate a candidate protein having a property of binding to a target protein and a protein motif that are specified by the user.

The target protein may be one or more proteins selected from among proteins associated with one or more of onset, treatment, prevention, and amelioration of a human disease.

The system may generate protein sequence information and a candidate protein corresponding to all or a part of an antibody or binding fragment thereof.

The system may generate protein sequence information including amino acid sequence information corresponding to a complementary binding region of the antibody or binding fragment thereof.

The structure guidance may be one or more selected from a group consisting of binding affinity to the target protein, immunogenicity to B cells, and off-target binding affinity.

The sequence guidance may be one or more selected from a group consisting of Immunogenicity to B cells and immunogenicity to helper T cells.

Another embodiment of the present disclosure may provide a protein sequence generation method performed by at least one processor.

The method may include a step of obtaining reference protein sequence information, a step of generating noise-added protein sequence information by repeatedly adding noise to the reference protein sequence information, and a step of generating noise-removed output protein sequence information from the noise-added protein sequence information, wherein the steps of the generating may include a step of generating the noise-removed protein sequence information by removing noise from input protein sequence information according to one or more protein structure guidances specified by a user, and a step of repeatedly performing all or part of a step of generating the noise-added protein sequence information by adding noise to the noise-removed protein sequence information according to one or more protein sequence guidances specified by the user, and

wherein all or part of the steps of the generating are repeatedly performed; and

a step of using the noise-removed outpt protein sequence information resulting from a final iteration of the generating steps to synthesize a candidate protein, wherein, in relation to the reference protein, the candidate protein exhibits improved properties corresponding to the protein structure guidances.

The method may generate a candidate protein having a property of binding to a target protein specified by the user.

The target protein may be one or more proteins selected from among proteins associated with one or more of onset, treatment, prevention, and amelioration of a human disease.

The method may generate protein sequence information and a candidate protein corresponding to all or a part of an antibody or binding fragment thereof.

The method may generate protein sequence information including amino acid sequence information corresponding to a complementary binding region of the antibody or binding fragment thereof.

The structure guidance may be one or more selected from a group consisting of binding affinity to the target protein, immunogenicity to B cells, and off-target binding affinity.

The sequence guidance may be one or more selected from a group consisting of immunogenicity to B cells and immunogenicity to helper T cells.

The present disclosure may provide a program stored in a computer-readable recording medium to execute the methods on a computer.

The method steps of generating noise-added protein sequence information may be carried out using a Gaussian noise model.

The method step(s) of generating noise-removed protein sequence information may be carried out using an artificial neural network model trained by a method of minimizing a loss function.

Protein sequence information for a protein having a user-desired property can be obtained.

Amino acid sequence information for therapeutic proteins that have excellent disease treatment effects and excellent human safety can be obtained, wherein the therapeutic proteins have a property of binding to a target protein specified by a user and have a protein motif specified by the user.

The costs and time required for new drug development can be significantly reduced by utilizing amino acid sequences of therapeutic proteins generated according to embodiments of the present disclosure.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention, and together with the description serve to explain the inventive concepts.

FIG. 1 is a flowchart showing a system and a method for generating output protein sequence information by repeating steps of adding noise to reference protein sequence information and then removing noise therefrom according to one embodiment of the present disclosure.

FIG. 2 is a flowchart showing a repetitive process of removing noise from noise-added protein sequence information and then adding noise again according to one embodiment of the present disclosure, and showing a step in which structure guidance and sequence guidance specified by a user are reflected in the repetitive process.

FIG. 3 is a block diagram showing an apparatus for generating protein sequence information from reference protein sequence information according to one embodiment of the present disclosure.

### DETAILED DESCRIPTION

The present disclosure fills the need in the art for an artificial intelligence system and method that are capable of predicting improved and viable candidate proteins for testing as therapeutics in a variety of health fields, thereby shortening the research and development time that has been necessary for advances in these fields.

In the following description, for the purposes of explanation, numerous specific details are set forth in order to provide a thorough understanding of various embodiments or implementations of the invention. As used herein “embodiments” and “implementations” are interchangeable words that are non-limiting examples of devices or methods employing one or more of the inventive concepts disclosed herein. It is apparent, however, that various embodiments may be practiced without these specific details or with one or more equivalent arrangements. In other instances, well-known structures and devices are shown in block diagram form in order to avoid unnecessarily obscuring various embodiments. Further, various embodiments may be different, but do not have to be exclusive. For example, specific shapes, configurations, and characteristics of an embodiment may be used or implemented in another embodiment without departing from the inventive concepts.

Unless otherwise specified, the illustrated embodiments are to be understood as providing features of varying detail of some ways in which the inventive concepts may be implemented in practice. Therefore, unless otherwise specified, the features, components, modules, layers, films, panels, regions, and/or aspects, etc. (hereinafter individually or collectively referred to as “elements”), of the various embodiments may be otherwise combined, separated, interchanged, and/or rearranged without departing from the inventive concepts.

The use of cross-hatching and/or shading in the accompanying drawings is generally provided to clarify boundaries between adjacent elements. As such, neither the presence nor the absence of cross-hatching or shading conveys or indicates any preference or requirement for particular materials, material properties, dimensions, proportions, commonalities between illustrated elements, and/or any other characteristic, attribute, property, etc., of the elements, unless specified. Further, in the accompanying drawings, the size and relative sizes of elements may be exaggerated for clarity and/or descriptive purposes. When an embodiment may be implemented differently, a specific process order may be performed differently from the described order. For example, two consecutively described processes may be performed substantially at the same time or performed in an order opposite to the described order. Also, like reference numerals denote like elements.

When an element, such as a layer, is referred to as being “on,” “connected to,” or “coupled to” another element or layer, it may be directly on, connected to, or coupled to the other element or layer or intervening elements or layers may be present. When, however, an element or layer is referred to as being “directly on,” “directly connected to,” or “directly coupled to” another element or layer, there are no intervening elements or layers present. To this end, the term “connected” may refer to physical, electrical, and/or fluid connection, with or without intervening elements. Further, the D1-axis, the D2-axis, and the D3-axis are not limited to three axes of a rectangular coordinate system, such as the x, y, and z – axes, and may be interpreted in a broader sense. For example, the D1-axis, the D2-axis, and the D3-axis may be perpendicular to one another, or may represent different directions that are not perpendicular to one another. For the purposes of this disclosure, “at least one of X, Y, and Z” and “at least one selected from the group consisting of X, Y, and Z” may be construed as X only, Y only, Z only, or any combination of two or more of X, Y, and Z, such as, for instance, XYZ, XYY, YZ, and ZZ. As used herein, the term “and/or” includes any and all combinations of one or more of the associated listed items.

Although the terms “first,” “second,” etc. may be used herein to describe various types of elements, these elements should not be limited by these terms. These terms are used to distinguish one element from another element. Thus, a first element discussed below could be termed a second element without departing from the teachings of the disclosure.

Spatially relative terms, such as “beneath,” “below,” “under,” “lower,” “above,” “upper,” “over,” “higher,” “side” (e.g., as in “sidewall”), and the like, may be used herein for descriptive purposes, and, thereby, to describe one elements relationship to another element(s) as illustrated in the drawings. Spatially relative terms are intended to encompass different orientations of an apparatus in use, operation, and/or manufacture in addition to the orientation depicted in the drawings. For example, if the apparatus in the drawings is turned over, elements described as “below” or “beneath” other elements or features would then be oriented “above” the other elements or features. Thus, the exemplary term “below” can encompass both an orientation of above and below. Furthermore, the apparatus may be otherwise oriented (e.g., rotated 90 degrees or at other orientations), and, as such, the spatially relative descriptors used herein interpreted accordingly.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof. It is also noted that, as used herein, the terms “substantially,” “about,” and other similar terms, are used as terms of approximation and not as terms of degree, and, as such, are utilized to account for inherent deviations in measured, calculated, and/or provided values that would be recognized by one of ordinary skill in the art.

Various embodiments are described herein with reference to sectional and/or exploded illustrations that are schematic illustrations of idealized embodiments and/or intermediate structures. As such, variations from the shapes of the illustrations as a result, for example, of manufacturing techniques and/or tolerances, are to be expected. Thus, embodiments disclosed herein should not necessarily be construed as limited to the particular illustrated shapes of regions, but are to include deviations in shapes that result from, for instance, manufacturing. In this manner, regions illustrated in the drawings may be schematic in nature and the shapes of these regions may not reflect actual shapes of regions of a device and, as such, are not necessarily intended to be limiting.

As is customary in the field, some embodiments are described and illustrated in the accompanying drawings in terms of functional blocks, units, and/or modules. Those skilled in the art will appreciate that these blocks, units, and/or modules are physically implemented by electronic (or optical) circuits, such as logic circuits, discrete components, microprocessors, hard-wired circuits, memory elements, wiring connections, and the like, which may be formed using semiconductor-based fabrication techniques or other manufacturing technologies. In the case of the blocks, units, and/or modules being implemented by microprocessors or other similar hardware, they may be programmed and controlled using software (e.g., microcode) to perform various functions discussed herein and may optionally be driven by firmware and/or software. It is also contemplated that each block, unit, and/or module may be implemented by dedicated hardware, or as a combination of dedicated hardware to perform some functions and a processor (e.g., one or more programmed microprocessors and associated circuitry) to perform other functions. Also, each block, unit, and/or module of some embodiments may be physically separated into two or more interacting and discrete blocks, units, and/or modules without departing from the scope of the inventive concepts. Further, the blocks, units, and/or modules of some embodiments may be physically combined into more complex blocks, units, and/or modules without departing from the scope of the inventive concepts.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and should not be interpreted in an idealized or overly formal sense, unless expressly so defined herein.

In order to clarify the technical spirit of the present disclosure, embodiments of the present disclosure will be described in detail with reference to the accompanying drawings. In describing the present disclosure, when it is determined that the detailed description of a related known function or component may unnecessarily obscure the gist of the present disclosure, the detailed description thereof will be omitted. In the drawings, components having substantially the same function or configuration are given the same reference numerals and symbols as possible even when they are shown in different drawings. For convenience of explanation, an apparatus and method will be described together when necessary. Each operation of the present disclosure do not necessarily need to be performed in the order described, and may be performed in parallel, selectively, or individually.

Terms used in the embodiments of the present disclosure were selected as general terms widely used at present as possible while considering functions of the present disclosure, but these terms may vary depending on the intention of those skilled in the art, legal precedents, the emergence of new technologies, or the like. In addition, in specific cases, there are terms arbitrarily selected by the applicant, and in this case, the meanings thereof will be described in detail in the description of the corresponding embodiment. Therefore, terms used in the present specification should be defined based on the meanings of the terms and the overall contents of the present disclosure rather than just the names of the terms.

Throughout the present disclosure, singular expressions may include plural expressions unless the context explicitly states otherwise. It should be understood that terms such as "comprise" or "have" are intended to specify the presence of a feature, number, step, operation, component, part, or a combination thereof, but do not preemptively preclude the possibility of the presence or addition of one or more other features, numbers, steps, operations, components, parts, or combinations thereof. That is, throughout the present disclosure, when a certain portion is described as “including,” a certain component, it means further including another component rather than precluding another component unless especially stated otherwise.

Expressions such as "at least one" modify the entire list of components, and do not individually modify components of the list. For example, "at least one of A, B, and C" or "at least one of A, B, or C" refers to only A, only B, only C, both A and B, both B and C, both A and C, all of A, B, and C, or a combination thereof.

In addition, terms such as "…unit," "…module", etc. described in the present disclosure mean a unit that process at least one function or operation, which may be implemented as hardware or software, or a combination of hardware and software.

The expression “configured to (or set to)” as used throughout the present disclosure may, depending on the contexts, be used interchangeably with, for example, “suitable for,” “having the capacity to,” “designed to,” “adapted to,” “made to,” or “capable of.” The term “configured to (or set to)” does not necessarily mean only “specifically designed to” in hardware. Instead, in certain contexts, the expression “a system configured to” may mean that the system is “capable of” in conjunction with other apparatuses or parts. For example, the phrase "a processor configured to (or set to) perform A, B, and C" may mean a dedicated processor (e.g., an embedded processor) for performing corresponding operations, or a generic-purpose processor (e.g., a CPU or application processor) that can perform corresponding operations by executing one or more software programs stored in memory.

Functions related to artificial intelligence according to the present disclosure are operated through the processor and the memory. The processor may include one or a plurality of processors. In this case, the one or plurality of processors may be a general-purpose processor such as a CPU, an AP, or a digital signal processor (DSP), a graphics-dedicated processor such as a graphics processing unit (GPU) or a vision processing unit (VPU), or an artificial intelligence-dedicated processor such as a neural processing unit (NPU). The one or plurality of processors may control input data to be processed according to a predefined operation rule or an artificial intelligence model that are stored in the memory. Alternatively, when the one or plurality of processors are artificial intelligence-dedicated processors, the artificial intelligence-dedicated processor may be designed with a hardware structure specialized for processing a specific artificial intelligence model.

The predefined operation rule or the artificial intelligence model is characterized by being created through training. Here, being created through learning means that the predefined operation rule or the artificial intelligence model is created by being trained with learning data by a learning algorithm, thereby setting the predefined operation rule or the artificial intelligence to achieve a desired objective. Such training may be performed on a device itself in which the artificial intelligence according to the present disclosure is performed, or may be performed through a separate server and/or system.

Throughout the present disclosure, the apparatus may include a server, a smartphone, a tablet PC, a PC, a TV, a smart TV, a mobile phone, a personal digital assistant (PDA), a speaker, a laptop, a media player, a micro server, an e-book object recognition apparatus, a digital broadcasting object recognition apparatus, a kiosk, an MP3 player, a digital camera, a robot vacuum cleaner, home appliances, other mobile or non-mobile computing apparatuses, or a watch, glasses, a hairband, or a ring that has a communication function and a data processing function, but is not limited thereto.

The present disclosure relates to a system and a method for generating protein sequence information using a noise-based diffusion model. The diffusion model includes a forward diffusion step of gradually adding noise perturbing data to protein data and a reverse denoising step of converting the noise-added data into noise-removed protein data. The diffusion model is trained as a deep learning model by parameterizing the reverse conversion step, and the trained deep learning model performs reverse conversion to generate the noise-removed data from the noise-added data.

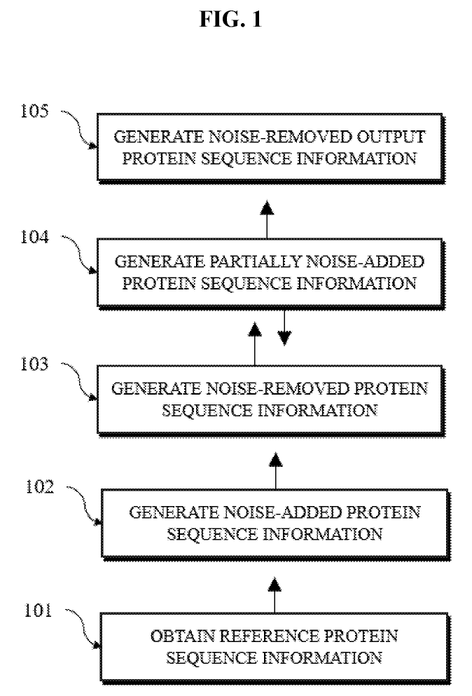

FIG. 1 shows a system and a method for generating output protein sequence information by repeating steps of adding noise to reference protein sequence information and then removing noise therefrom using a diffusion model according to one embodiment of the present disclosure.

Obtaining reference protein sequence information (101)

The reference protein sequence information refers to information about amino acid sequences of reference proteins. According to one embodiment of the present disclosure, the reference protein sequence information uses information extracted from amino acid sequences of previously known proteins. According to one embodiment of the present disclosure, the amino acid sequences of proteins forming a basis of the reference protein sequence information may be selected from amino acid sequences of proteins known to have a protein motif specified by a user while binding to a target protein specified by the user.

Generating noise-added protein sequence information (102)

According to one embodiment of the present disclosure, noise-added protein sequence information may be generated by a diffusion step of gradually adding noise perturbing data to the obtained reference protein sequence information.

According to one embodiment of the present disclosure, one or more noise models selected from a group consisting of Gaussian noise, salt-and-pepper noise, Poisson noise, binomial noise, and speckle noise may be used, and depending on applications of the diffusion model, the noise model may be appropriately selected, or two or more noise models may be combined to construct the system or the method, and the user may specify types of noise models used in the system and the method of the present disclosure. In one embodiment of the present disclosure, preferably, a Gaussian noise model is used, but the present disclosure is not limited thereto.

According to one embodiment of the present disclosure, the diffusion step includes a step of gradually adding noise over a plurality of steps. According to one embodiment of the present disclosure, noise is added to the reference protein sequence information, and noise is repeatedly added to the noise-added information to finally generate the noise-added protein sequence information.

Generating noise-removed protein sequence information (103)

According to one embodiment of the present disclosure, protein sequence information predicted to have a user-desired property may be generated by removing noise from the noise-added protein sequence information using an artificial neural network.

According to one embodiment of the present disclosure, the artificial neural network may remove noise from the noise-added protein sequence information to generate noise-removed protein sequence information, and a protein having an amino acid sequence extracted from the noise-removed protein sequence information may be predicted to have a protein structure that gives that protein the user-desired property.

According to one embodiment of the present disclosure, a step of generating the noise-removed protein sequence information by removing noise from the noise-added protein sequence information may be performed using an artificial neural network model trained by a method of minimizing a loss function. According to one embodiment of the present disclosure, the step of removing noise may include doing so via a plurality of steps, thereby gradually removing the noise.

Generating noise-added protein sequence information (104)

By repeatedly performing a step of adding noise again to the noise-removed protein sequence information (104, 221) and then a step of removing noise again (103, 211), protein sequence information providing a protein with high reliability of having a user-desired property may be obtained.

According to one embodiment of the present disclosure, the same noise model as used in the diffusion step of adding noise to the reference protein sequence information (102) may be used to add noise to the noise-removed protein sequence information, and the Gaussian noise model may be used as the noise model, but the present disclosure is not limited thereto.

Generating noise-removed output protein sequence information (105)

By repeatedly performing a step of removing noise from the noise-added protein sequence information (103, 211) and a step of adding noise again to the noise-removed protein sequence information (104, 221), output protein sequence information providing a protein with high reliability of having a user-desired property may be obtained.

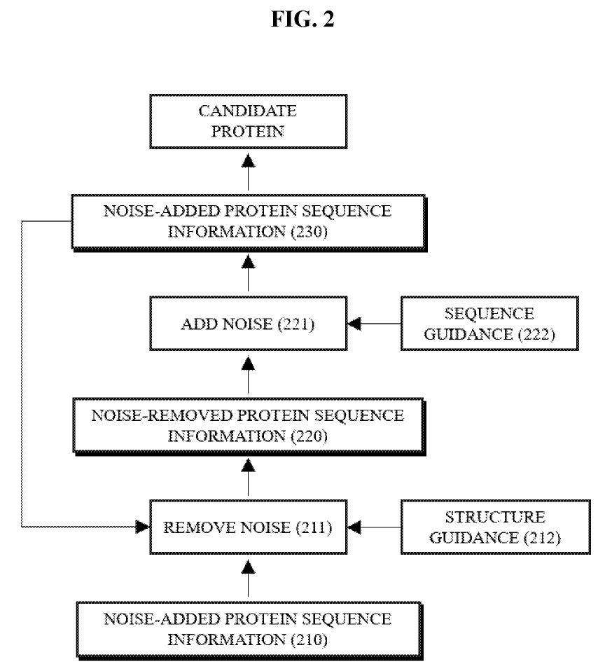

FIG. 2 is a flowchart specifically showing a denoising step and steps of the FIG. 1 flowchart in which structure guidance and sequence guidance are applied.

Step of removing noise in the denoising step (211)

According to one embodiment of the present disclosure, a step of generating the noise-removed protein sequence information by removing noise from the noise-added protein sequence information may be performed using an artificial neural network model trained by a method of minimizing a loss function.

According to one embodiment of the present disclosure, in the step of removing noise (211), an artificial neural network trained to generate protein sequence information predicted to have properties according to the structure guidance may be used.

According to one embodiment of the present disclosure, properties associated with structures of proteins may be used as the structure guidance. The structure guidance may be one or more selected from a group consisting of binding affinity to the target protein, immunogenicity to B cells, and off-target binding affinity.

The binding affinity to the target protein refers to a property that a therapeutic protein binds to the target protein, and the higher the binding affinity to the target protein, the greater the amount bound to the target protein even when a small amount of the therapeutic protein is applied. It may be predicted that the higher the binding affinity to the target protein, the greater the disease treatment effect of the therapeutic protein.

Immunogenicity to B cells means a property that a therapeutic protein causes an immune response to be generated in a human body by B cells. When the therapeutic protein causes an excessive immune response to be generated by B cells after the therapeutic protein is administered, fatal side effects may occur in a human body, and, therefore, it may be predicted that the lower the immunogenicity to B cells, the higher the human safety of the therapeutic protein.

Off-target binding affinity refers to the tendency of a therapeutic protein to bind in a human body to biomacromolecules (such as proteins) other than the target protein. When the therapeutic protein binds to other biomacromolecules in a human body, not the target protein, after being administered into the human body, side effects are more likely to occur, and therefore, it may be predicted that the lower the off-target binding affinity, the higher the human safety of the therapeutic protein.

According to one embodiment of the present disclosure, the artificial neural network may generate amino acid sequence information for proteins having properties set by the user as the structure guidance by removing noise from the noise-added sequence information data.

Step of adding noise (221)

By repeatedly performing a step of adding noise again (221) to the noise-removed protein sequence information and then a step of removing noise again (211), protein sequence information providing proteins reliably featuring a user-desired property may be obtained.

According to one embodiment of the present disclosure, the same noise model as used in the diffusion step (102) of adding noise to the reference protein sequence information may be used to later add noise to the noise-removed protein sequence information. The Gaussian noise model may be used as the noise model, but the present disclosure is not limited thereto.

According to one embodiment of the present disclosure, properties associated with protein sequences of known proteins may be used as the sequence guidance. The sequence guidance may be one or more selected from a group consisting of immunogenicity to B cells and immunogenicity to helper T cells.

The helper T cells are immune cells that may be activated by fragmented external proteins (therapeutic proteins), and immunogenicity to helper T cells may be determined to depend on amino acid sequences constituting the fragmented external proteins. When the therapeutic protein includes protein amino acid sequences capable of activating helper T cells, immunogenicity to helper T cells may be high, and in this case, the therapeutic protein may excessively cause unwanted immune responses, thereby leading to fatal side effects. Therefore, it may be predicted that the lower the immunogenicity to helper T cells, the higher the human safety of the therapeutic protein.

The B cells are activated by binding to structures of the therapeutic proteins, and, in this case, the protein sequences and the secondary, tertiary and quaternary structures of the therapeutic proteins may affect whether the B cells are activated. Therefore, according to one embodiment of the present disclosure, structural information about proteins known to exhibit immunogenicity to B cells may be used as the sequence guidance as well as the structure guidance.

According to one embodiment of the present disclosure, by repeatedly performing steps of adding noise to and then removing noise from noise-removed sequence information data, amino acid sequence information for proteins having properties set by the user as the sequence guidance may be generated.

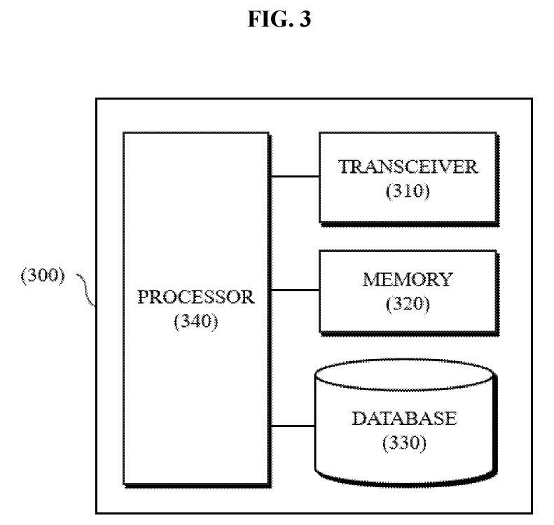

FIG. 3 is a block diagram of a protein representation learning apparatus according to one embodiment of the present disclosure.

Referring to FIG. 3, a protein representation learning apparatus 300 may include a transceiver 310, a memory 320, a database 330, and a processor 340. However, not all of the components shown in FIG. 3 are essential components of the protein representation learning apparatus 300. The protein representation learning apparatus 300 may be implemented with more components than those shown in FIG. 3, or the protein representation learning apparatus 300 may be implemented with fewer components than those shown in FIG. 3. In addition, the transceiver 310, the memory 320, and the processor 340 may be implemented in the form of a single chip.

In one embodiment, the transceiver 310 may communicate with a terminal or other electronic devices connected to the protein representation learning apparatus 300 in a wired or wireless communication manner. For example, the transceiver 310 may obtain amino acid sequence information of proteins, protein interaction data, or protein representations generated using an artificial neural network, or the like from other electronic devices.

The memory 320 may install and store various types of data such as programs and files including applications. The processor 340 may access data stored in the memory 320 and use the data, or may store new data in the memory 320. In addition, the memory 320 may store one or more instructions. The processor 340 may execute the one or more instructions stored in the memory.

The processor 340 may control the overall operation of the protein representation learning apparatus 300 and may include at least one processor such as a central processing unit (CPU), a graphics processing unit (GPU), and the like. The processor 340 may control other components included in the protein representation learning apparatus 300 to perform operations of the protein representation learning apparatus 300. For example, the processor 340 may obtain protein data, obtain protein representations using the neural network, calculate a contrastive loss from the protein representations, and modify one or more values of one or more parameters of one or more encoder neural networks based on the contrastive loss.

The database 330 may store various training data for training a learning model. In addition, the database 330 may store amino acid sequence information of proteins, protein interaction data, protein structure information, simulation result information, and the like, and, in various embodiments, the database 330 may also store output data generated by the learning model. In FIG. 3, the protein representation learning apparatus 300 is illustrated as including the database 330, but the database 330 may be provided outside the apparatus. In this case, the database 330 may be connected to the protein representation learning apparatus 300 in a wired or wireless communication manner.

In addition, the learning model may be implemented outside the protein representation learning apparatus 300 (e.g., implemented in a cloud-based environment), or may be included in the protein representation learning apparatus 300.

One embodiment of the present disclosure may also be implemented in the form of a recording medium including computer-executable instructions such as program modules executed by a computer. A computer-readable medium may be any available medium that can be accessed by the computer, and may include all of volatile and non-volatile media, and removable and non-removable media. In addition, the computer-readable medium may include both computer storage media and communication media. The computer storage media may include all of volatile and non-volatile, removable and non-removable media that are implemented in any method or technology for storage of information such as computer-readable instructions, data structures, program modules, or other data. The communication media typically may include computer-readable instructions, data structures, or program modules and may include any information delivery media.

The protein sequence information generation system of the present disclosure may be used for various purposes such as searching for candidate substances for new drug development.

For example, the user may input a dataset including information about amino acid sequences of proteins, binding affinities to targets, and the like into the protein sequence information generation system of the present invention, thereby obtaining amino acid sequence information of proteins that have properties specified by the user of binding to the target protein and have the protein motif specified by the user. The user may synthesize proteins according to the protein amino acid sequences obtained through the protein sequence information generation system of the present disclosure and may obtain therapeutic proteins having high biological safety and low possibility of causing immune responses, thereby obtaining therapeutic proteins that are suitable for roles as candidate substances for new drug development.

The protein sequence information generation system of the present disclosure considers immunogenicity, toxicity, stability, and structural folding, thereby allowing elimination in advance of candidates highly likely to be dropped in preclinical stages and enabling preferential development of safe and effective protein drugs, and, therefore, significant reduction in costs and time for new drug development may be expected.

The above description of the present disclosure is for illustrative purposes, and those skilled in the art to which the present disclosure pertains will understand that various modifications can be easily made into other specific forms without departing from the technical spirit or essential characteristics of the present invention. Therefore, it should be understood that the above-described embodiments are illustrative and not restrictive in all respects. For example, each component described in a singular form may be implemented separately, and likewise, components described as being implemented separately may also be implemented in a combined form.

Although certain embodiments and implementations have been described herein, other embodiments and modifications will be apparent from this description. Accordingly, the inventive concepts are not limited to such embodiments, but rather to the broader scope of the appended claims and various obvious modifications and equivalent arrangements as would be apparent to a person of ordinary skill in the art.

WHAT IS CLAIMED IS:

1. A protein sequence generation system for new therapeutic protein drug development, the system comprising:
equipment for synthesizing a protein;
a memory configured to store one or more instructions; and
at least one processor configured to execute the one or more instructions stored in the memory,
wherein operations performed by the one or more instructions comprise:
a step of obtaining reference protein sequence information;
a step of generating noise-added protein sequence information by repeatedly adding noise to the reference protein sequence information; and
a step of generating noise-removed output protein sequence information from the noise-added protein sequence information,
wherein the steps of the generating comprise:
a step of generating the noise-removed protein sequence information by removing noise from input protein sequence information according to one or more protein structure guidances specified by a user; and
a step of repeatedly performing all or part of a step of generating the noise-added protein sequence information by adding noise to the noise-removed protein sequence information according to one or more protein sequence guidances specified by the user,
wherein all or part of the steps of the generating are repeatedly performed,
wherein the noise-removed output protein sequence information resulting from a final iteration of the generating steps is used with the equipment to synthesize a candidate protein, and
wherein, in relation to the reference protein, the candidate protein exhibits improved properties corresponding to the protein structure guidances.

2. The system according to claim 1, wherein
the system generates a candidate protein having a property of binding to a target protein and a protein motif that are specified by the user.

3. The system according to claim 2, wherein
the target protein is one or more proteins selected from among proteins associated with one or more of onset, treatment, prevention, and amelioration of a human disease.

4. The system according to claim 3, wherein
the system generates protein sequence information and a candidate protein corresponding to all or a part of an antibody or binding fragment thereof.

5. The system according to claim 4, wherein
the system generates protein sequence information including amino acid sequence information corresponding to a complementary binding region of the antibody or binding fragment thereof.

6. The system according to claim 2, wherein
the structure guidance is one or more selected from a group consisting of binding affinity to the target protein, immunogenicity to B cells, and off-target binding affinity.

7. The system according to claim 2, wherein
the sequence guidance is one or more selected from a group consisting of immunogenicity to B cells and immunogenicity to helper T cells.

8. A protein sequence generation method performed by at least one processor, the method comprising:
a step of obtaining reference protein sequence information;
a step of generating noise-added protein sequence information by repeatedly adding noise to the reference protein sequence information; and
a step of generating noise-removed output protein sequence information from the noise-added protein sequence information,
wherein the steps of the generating comprise:
a step of generating the noise-removed protein sequence information by removing noise from input protein sequence information according to one or more protein structure guidance specified by a user; and
a step of repeatedly performing all or part of a step of generating the noise-added protein sequence information by adding noise to the noise-removed protein sequence information according to one or more protein sequence guidances specified by the user, and
wherein all or part of the steps of the generating are repeatedly performed; and
a step of using the noise-removed output protein sequence information resulting from a final iteration of the generating steps to synthesize a candidate protein,
wherein, in relation to the reference protein, the candidate protein exhibits improved properties corresponding to the protein structure guidances.

9. The method according to claim 8, wherein
the method generates a candidate protein having a property of binding to a target protein specified by the user.

10. The method according to claim 9, wherein
the target protein is one or more proteins selected from among proteins associated with one or more of onset, treatment, prevention, and amelioration of a human disease.

11. The method according to claim 10, wherein
the method generates protein sequence information and a candidate protein corresponding to all or a part of an antibody or binding fragment thereof.

12. The method according to claim 11, wherein
the method generates protein sequence information including amino acid sequence information corresponding to a complementary binding region of the antibody or binding fragment thereof.

13. The method according to claim 9, wherein
the structure guidance is one or more selected from a group consisting of binding affinity to the target protein, immunogenicity to B cells, and off-target binding affinity.

14. The method according to claim 9, wherein
the sequence guidance is one or more selected from a group consisting of immunogenicity to B cells and immunogenicity to helper T cells.

15. A program stored in a computer-readable recording medium to execute the method according to claim 8 on a computer.

16. The method according to claim 8, wherein the steps of generating noise-added protein sequence information are carried out using a Gaussian noise model.

17. The method according to claim 8, wherein the step(s) of generating noise-removed protein sequence information are carried out using an artificial neural network model trained by a method of minimizing a loss function.
